(19) United States
(12) Patent Application Publication
(10) Pub. No.: US 2019/0015598 A1
(43) Pub. Date: Jan. 17, 2019

(54) SYRINGE ASSEMBLY, SYRINGE ASSEMBLY PACKAGING, AND PREFILLED SYRINGE

(71) Applicant: TERUMO KABUSHIKI KAISHA, Tokyo (JP)

(72) Inventors: Masafumi TAKEMOTO, Shizuoka (JP); Hitoshi OKIHARA, Shizuoka (JP)

(73) Assignee: TERUMO KABUSHIKI KAISHA, Tokyo (JP)

(21) Appl. No.: 16/133,321

(22) Filed: Sep. 17, 2018

Related U.S. Application Data
(63) Continuation of application No. PCT/JP2016/058616, filed on Mar. 17, 2016.

(51) Int. Cl. A61M 5/32; A61J 1/14

(52) U.S. Cl. CPC A61M 5/3202; A61J 1/14; A61J 1/06; A61M 5/329; A61M 5/3245

(57) ABSTRACT

A syringe assembly includes: a barrel with a puncture needle; and a cap that is mounted on the barrel and in which a tip of the puncture needle 6 is located. The cap includes: a seal cap that is made of an elastic material and that is water vapor permeable for autoclave sterilization; and a cylindrical cover member made of a thermoplastic plastic material that is harder than the seal cap. The cylindrical cover member and the seal cap are configured such that a force of engagement between the cylindrical cover member and the seal cap is increased by autoclave sterilization.

### CROSS REFERENCE TO RELATED APPLICATIONS

The present application is a bypass continuation of PCT Application No. PCT/JP2016/058616, filed on Mar. 17, 2016, which is hereby incorporated by reference in its entirety.

### BACKGROUND

The present disclosure relates to a syringe assembly to which an external cylinder seal cap is mounted, a prefilled syringe that includes syringe assembly to which an external cylinder seal cap is mounted, and a packaging storing a plurality of syringe assemblies.

A syringe having a puncture needle fixed at a distal end of an external cylinder is used as a syringe for administering a low dose of medical solution, such as an insulin syringe. When a prefilled syringe in which a medical solution is prefilled includes this type of syringe, a tip needs to be sealed. Such a seal cap capable of sealing the tip is proposed in, for example, U.S. Pat. No. 6,719,732.

### SUMMARY

A seal cap (a device that protects a syringe needle) of U.S. Pat. No. 6,719,732 includes an elastic cap 20 and a rigid shell 80 mounted to the cap 20 as illustrated in FIGS. 6 to 10 of U.S. Pat. No. 6,719,732. It is difficult for a needle 106 to pierce the shell 80. A longitudinal wall 88 of the shell 80 also includes four cut-outs 94 intended to allow passage of water vapor. In addition, the shell 80 includes a cap retaining means for retaining the cap 20 on an inner surface. As the retaining means, a protruding portion 96 protruding inwardly of a cavity is provided.

Typically, when the cap constituted by the rigid shell and the elastic cap as described above is detached from the syringe, the rigid shell and the seal cap are detached together. However, because the seal cap is in close contact with the syringe, when the cap is detached, there is a risk that a hard cover is detached from the seal cap and only the seal cap remains in the state of being mounted to an external cylinder. In particular, when high-pressure steam sterilization (autoclave sterilization) is performed, the seal cap is more likely to be fixed by the syringe due to heat during sterilization, and the above-described risk increases.

Therefore, an object of certain embodiments described in the present disclosure is to provide a syringe assembly to which an external cylinder seal cap, a prefilled syringe that includes a syringe assembly to which an external cylinder seal cap is mounted, and a packaging storing a plurality of syringe assemblies which can reliably remove the entire cap even when being stored in a state in which the seal cap is mounted to an external cylinder and even in the case of performing sterilization accompanied with heating such as high-pressure steam sterilization on the syringe assembly to which the seal cap is mounted.

In one embodiment, a syringe assembly includes: an external cylinder that includes an external cylinder body portion, a nozzle portion disposed at a distal end of the barrel body portion, and a puncture needle including a puncture needle tip at a distal end of the puncture needle and having a proximal end fixedly inserted into the nozzle portion; and a cap mounted to the barrel and sealing the puncture needle tip. The cap includes: a seal cap that includes a closed distal end portion, an open proximal end portion, a hollow portion including a puncture needle storage portion storing the puncture needle, an insertion-allowing portion to which the puncture needle tip of the puncture needle stored in the puncture needle storage portion is insertable, and a seal portion in close contact with the nozzle portion to seal the puncture needle storage portion; and a cylindrical cover member mounted at an outer side of the seal cap to be engaged with the seal cap. The seal cap is made of an elastic material and includes water vapor permeable properties for high-pressure steam sterilization. The cylindrical cover member is made of a thermoplastic plastic material harder than the seal cap, and a force of engagement between the cylindrical cover member and the seal cap is increased compared to before sterilization by autoclave sterilization of the syringe assembly.

In another embodiment, a prefilled syringe includes: the above-described syringe assembly; a gasket stored in the barrel and slidable in the barrel in a liquid-tight manner; and a medical solution filled in a space formed by the barrel and the gasket.

In another embodiment, a syringe assembly packaging storing a plurality of the above syringe assemblies includes: a container having an open upper surface and having shape retainability; an external cylinder holding member capable of holding the plurality of syringe assemblies; the plurality of syringe assemblies held by the barrel holding member; and a sheet-shaped lid member that hermetically seals the open upper surface of the container and is peelable. Further, the packaging includes a ventilation portion that is disposed on the container or the lid member and includes bacterial impermeable properties and sterilization gas flowability, and subjected to high-pressure steam sterilization.

### BRIEF DESCRIPTION OF THE DRAWINGS

FIG. 1 is a front view of a prefilled syringe according to an embodiment of the present invention.

FIG. 2 is a right side view of the prefilled syringe of FIG. 1.

FIG. 3 is a cross-sectional view taken along line A-A of FIG. 1.

FIG. 4 is a partially-omitted enlarged cross-sectional view of a syringe assembly of the present invention used in the prefilled syringe of FIGS. 1 and 2.

FIG. 5 is an enlarged front view of an external cylinder seal cap of the present invention used for the prefilled syringe of FIGS. 1 to 3 and the syringe assembly of FIG. 4.

FIG. 6 is a cross-sectional view taken along line B-B of FIG. 5.

FIG. 7 is an enlarged right side view of the cylindrical cover member used for the prefilled syringe illustrated in FIGS. 1 to 3 and the syringe assembly of FIG. 4.

FIG. 8 is a longitudinal cross-sectional view of the cylindrical cover member of FIG. 7.

FIG. 9 is a cross-sectional view taken along line C-C of FIG. 7.

FIG. 10 is an enlarged plan view of the cylindrical cover member of FIG. 7.

FIG. 11 is an enlarged bottom view of the cylindrical cover member of FIG. 7.

FIG. 12 is a perspective view of the cylindrical cover member illustrated in FIG. 7.

FIG. 13 is an enlarged front view of a seal cap used for the prefilled syringe illustrated in FIGS. 1 to 3 and the syringe assembly of FIG. 4.

FIG. 14 is a longitudinal cross-sectional view of the seal cap of FIG. 13.

FIG. 15 is a bottom view of the seal cap illustrated in FIG. 13.

FIG. 16 is a front view of an external cylinder with a puncture needle, which is used for the prefilled syringe illustrated in FIGS. 1 to 3 and the syringe assembly of FIG. 4.

FIG. 17 is a perspective view of the barrel with a puncture needle illustrated in FIG. 16.

FIG. 18 is a perspective view of a syringe assembly packaging of the present invention.

FIG. 19 is an explanatory view for describing an internal form of the syringe assembly packaging illustrated in FIG. 18.

FIG. 20 is a front view of the syringe assembly packaging illustrated in FIG. 18.

FIG. 21 is a plan view of the syringe assembly packaging illustrated in FIG. 20.

FIG. 22 is an enlarged cross-sectional view taken along line D-D of FIG. 21.

### DETAILED DESCRIPTION

A syringe assembly to which an external cylinder seal cap is mounted and a prefilled syringe that includes a syringe assembly to which an external cylinder seal cap is mounted according to embodiments of the present invention will be described with reference to embodiments illustrated in the drawings.

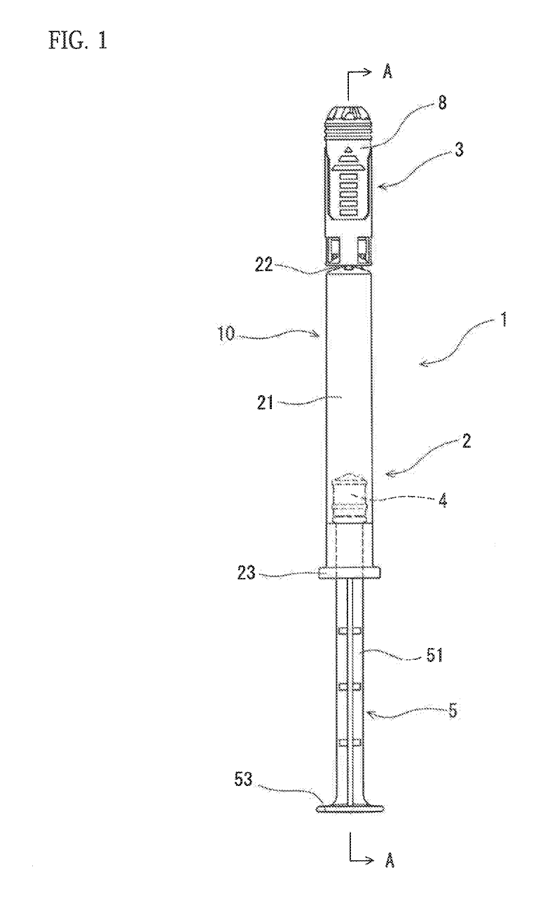

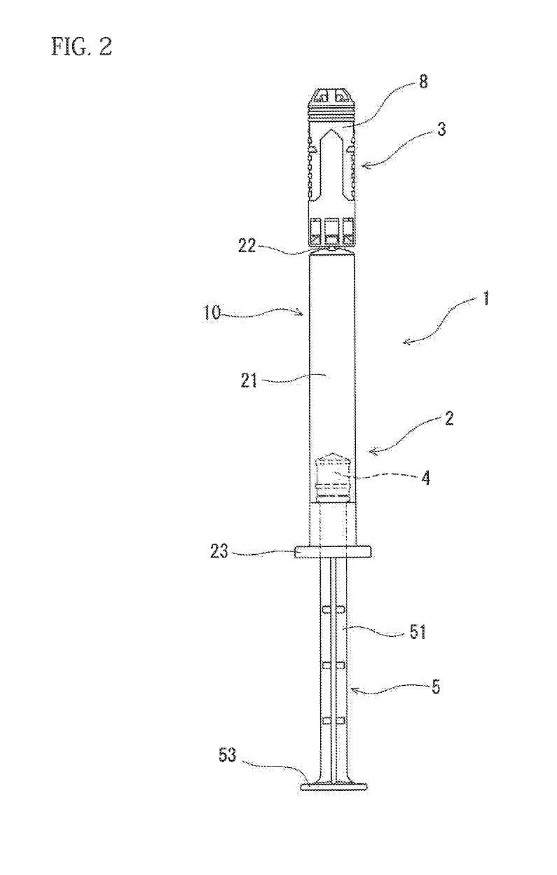

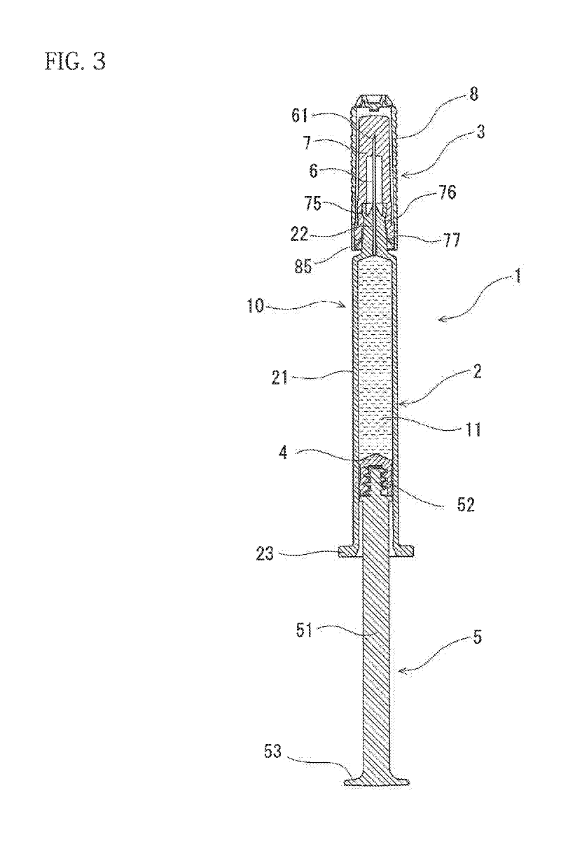

As illustrated in FIGS. 1 to 3, a prefilled syringe 1 according to one embodiment includes a syringe assembly 10, a gasket 4 stored in the syringe assembly 10 and slidable in the syringe assembly 10 in a liquid-tight manner, and a medical solution 11 filled in a space formed of the syringe assembly 10 and the gasket 4.

Further, the syringe assembly 10 according to the present embodiment (in other words, an external cylinder with a puncture needle, to which a cap is mounted) includes an external cylinder 2 and a cap 3 mounted to the barrel 2, as illustrated in FIGS. 1 to 4.

The barrel 2 includes an external cylinder body portion 21, a nozzle portion 22 disposed at a distal end of the barrel body portion 21, and a puncture needle 6 including a puncture needle tip 61 at a distal end thereof and a proximal end inserted into and fixed to the nozzle portion 22.

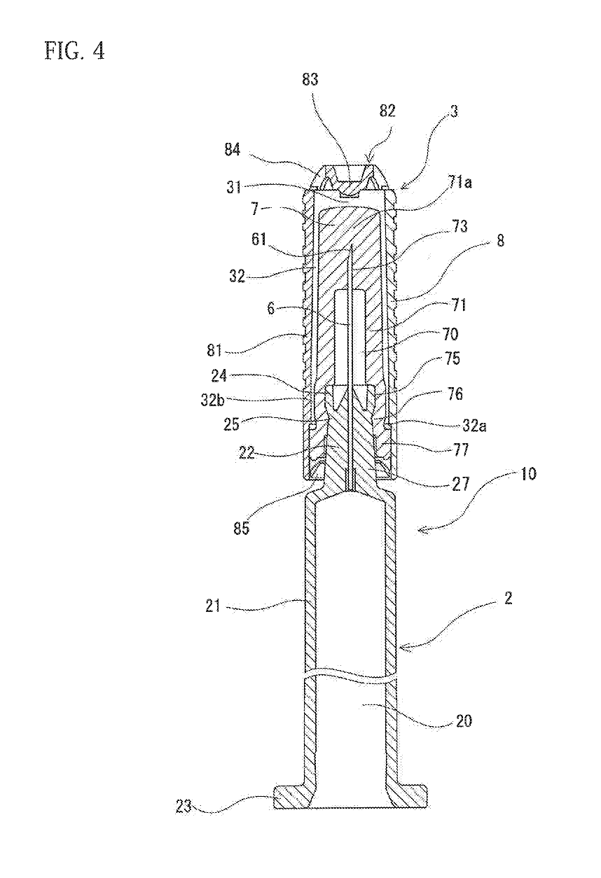

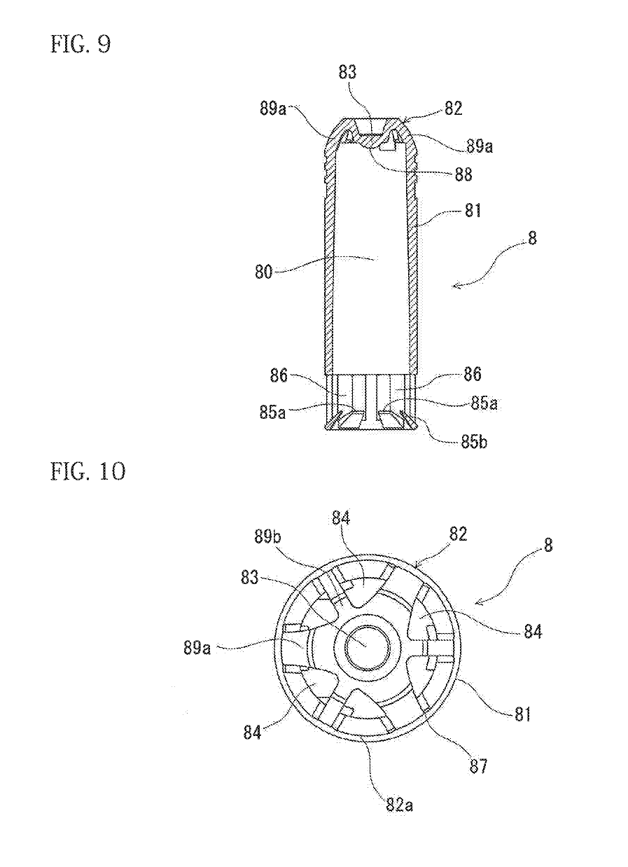

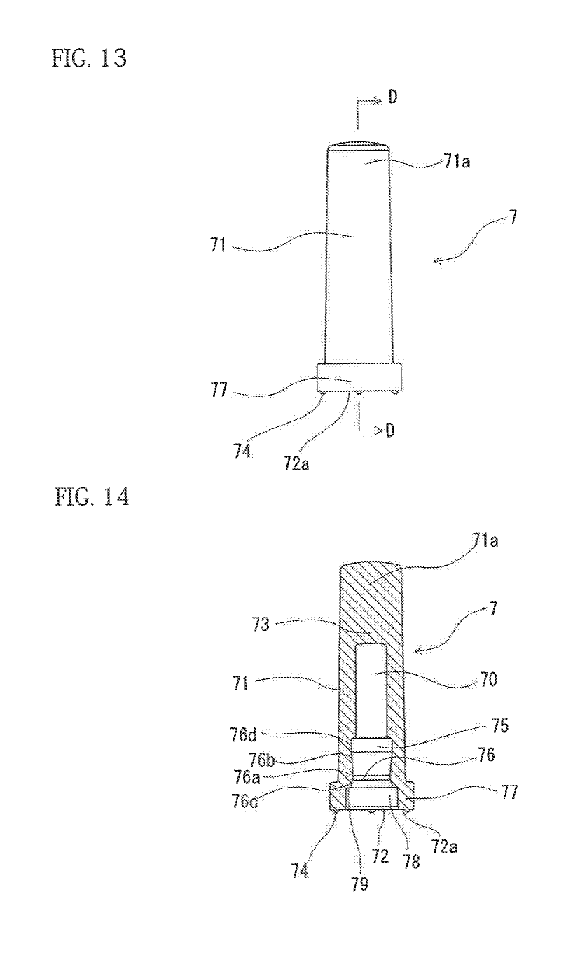

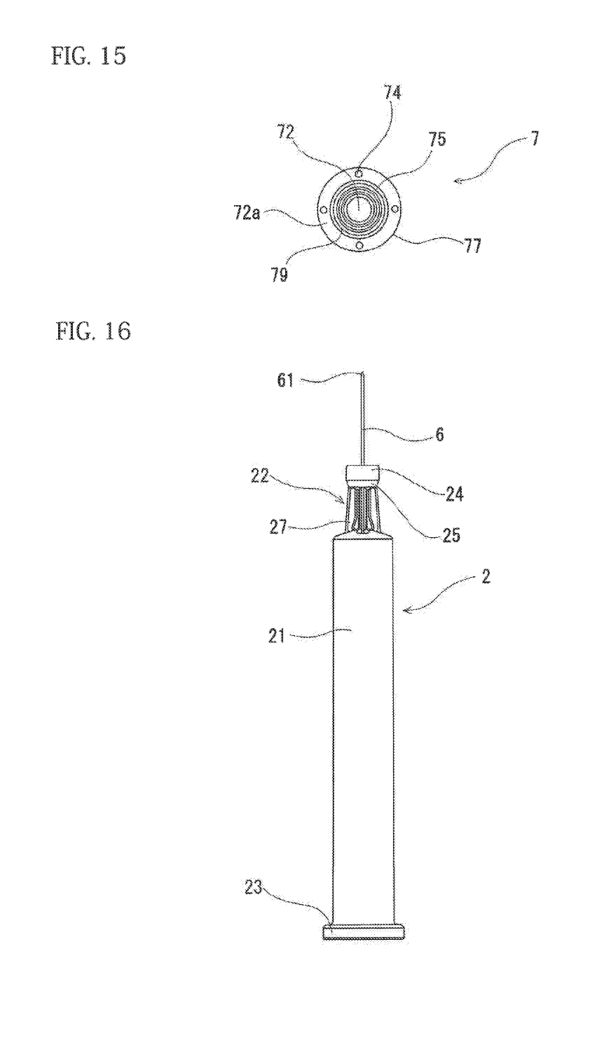

As illustrated in FIGS. 4 to 15, the cap 3 includes: a seal cap 7 which includes: a closed distal end portion 71a, an open proximal end portion 72, a hollow portion 70 including a puncture needle storage portion that stores the puncture needle 6, an insertion-allowing portion 73 into which the puncture needle tip 61 of the puncture needle 6 stored in the puncture needle storage portion is insertable, and a seal portion 75 in close contact with the nozzle portion to seal the puncture needle storage portion; and a cylindrical cover member 8 mounted at an outer side of the seal cap 7 to be engaged with the seal cap 7.

Further, the seal cap 7 is made of an elastic material and is permeable to water vapor (e.g., permeable to steam) for high-pressure steam sterilization (e.g., autoclave sterilization). The cylindrical cover member 8 is made of a thermoplastic material harder than the seal cap 7. The cap 3 is configured so that, by high-pressure steam sterilization of the syringe assembly 10, a force of engagement between the cylindrical cover member 8 and the seal cap 7 is increased compared to before sterilization.

As illustrated in FIGS. 1 to 3, the prefilled syringe 1 includes: the syringe assembly 10 including the barrel 2 and the cap 3 mounted to the barrel 2 so as to seal the tip of the puncture needle; the gasket 4 stored in the syringe assembly 10 and slidable in the syringe assembly 10 in a liquid-tight manner; the medical solution 11 filled in the space formed by the syringe assembly 10 and the gasket 4; and a plunger 5 attached to the gasket 4 or to be attached at the time of use.

Further, the medical solution 11 is filled in the space formed by the barrel 2, the gasket 4, and the cap 3.

As the medical solution 11 to be filled, any kind of medical solution may be used, and examples thereof may include a high concentration sodium chloride injection solution, minerals, a heparin sodium solution, nitroglycerin, isosorbide dinitrate, cyclosporine, benzodiazepines, antibiotics, vitamin preparations (multivitamin preparations), various amino acids, antithrombotic drugs such as heparin, insulin, antitumor drugs, analgesics, cardiotonics, intravenous anesthetics, an antiparkinsonism drugs, tumor therapeutic drugs, adrenal corticosteroids, arrhythmia drugs, correction electrolytes, antiviral drugs, and immunostimulants.

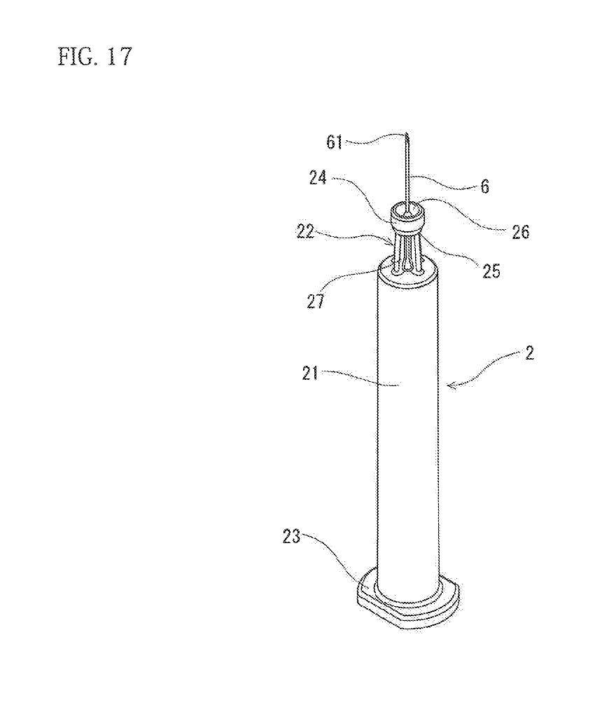

As illustrated in FIGS. 1 to 4, 16, and 17, the barrel 2 includes the barrel body portion 21, the cylindrical (hollow) nozzle portion 22 disposed at the distal end of the barrel body portion 21, a flange 23 disposed at a proximal end of the barrel body portion 21, and the puncture needle 6 having the proximal end inserted into and fixed to the nozzle portion 22. The puncture needle 6 includes the puncture needle tip 61 at the distal end thereof. The proximal end of the puncture needle 6 is inserted into and fixed to the hollow portion of the nozzle portion 22 and the inside of the puncture needle 6 communicates with an internal space 20 of the barrel 2. The puncture needle 6 may be inserted into the hollow portion of the nozzle portion 22 of the barrel 2 molded in advance and fixed to the nozzle portion 22 using an adhesive, heat welding, or the like. In addition, the puncture needle 6 may be directly fixed to the barrel 2 by insert-molding. In the case of insert-molding, the nozzle portion 22 forms a cylindrical shape (hollow shape) into which the puncture needle 6 has been inserted by molding the barrel 2, and the puncture needle 6 has the proximal end inserted into and fixed to the hollow portion of the nozzle portion 22.

The barrel 2 is transparent or translucent. The barrel body portion 21 is a substantially cylindrical portion that stores the gasket 4 in a liquid-tight and slidable manner. In addition, the nozzle portion 22 protrudes forward from a distal end (shoulder portion) of the barrel body portion and forms a hollow cylindrical shape with a smaller diameter than the barrel body portion. In addition, as illustrated in FIGS. 4, 16, and 17, the nozzle portion 22 includes a head portion 24 disposed at a distal end (in the present embodiment, an annular head portion whose inside is recessed), a short tapered reduced diameter portion 25 disposed at a proximal end of the head portion 24 and having a diameter reduced in a proximal end direction, and a connecting portion 27 connecting a proximal end of the tapered reduced diameter portion 25 and a distal end of the barrel body portion 21, and an annular concave portion is formed by the tapered reduced diameter portion 25.

A recess 26 recessed from a distal end surface toward a proximal end side and a hollow conical portion positioned in the recess 26 and including a vertex on a distal end side are formed in the head portion 24. A plurality of grooves extending in an axial direction of the barrel 2 is formed on an outer surface of the connecting portion 27. The annular concave portion may have a shape which is simply reduced in diameter so that a step is formed between the annular concave portion and the proximal end of the head portion 24, instead of the tapered shape. In addition, the connecting portion 27 may be omitted, and a proximal end of the annular concave portion (the tapered reduced diameter portion 25) and the distal end of the barrel body portion 21 may be directly connected. In addition, the head portion 24 may have a hollow columnar shape (cylindrical shape) in which the recess 26 and the conical portion are omitted.

Examples of a material for forming the barrel 2 may include various resins such as polypropylene, polyethylene, polystyrene, polyamide, polycarbonate, polyvinyl chloride, poly-(4-methylpentene-1), an acryl resin, an acrylonitrile-butadiene-styrene copolymer, polyester such as polyethylene terephthalate, and a cyclic polyolefin polymer, and a cyclic olefin copolymer. Among them, the polypropylene, the cyclic polyolefin (for example, the cyclic olefin polymer and the cyclic olefin copolymer) are preferable because these resins can be easily molded and are heat-resistant.

As the puncture needle 6, a hollow needle including the puncture needle tip 61 at a distal end is used. Metal is generally used as a material for forming the puncture needle 6. As the metal, stainless steel is preferable.

As illustrated in FIGS. 1 to 3, the gasket 4 includes a body portion extending with substantially the same outer diameter, and a plurality of annular ribs disposed on the body portion (two ribs in the present embodiment, and an appropriate number of ribs is set as long as the number satisfies the liquid-tightness and the slidability if the number is two or more), and these ribs are brought into contact with the inner surface of the barrel 2 in a liquid-tight manner. In addition, a distal end surface of the gasket 4 has a shape corresponding to a shape of a distal end inner surface of the barrel 2 so as not to form a gap as much as possible between the gasket 4 and the barrel when abutting against the distal end inner surface of the barrel 2.

As a material for forming the gasket 4, it is preferable to use elastic rubber (for example, isoprene rubber, butyl rubber, latex rubber, silicone rubber, or the like), a synthetic resin (for example, a styrene elastomer such as an SBS elastomer and an SEBS elastomer, an olefin elastomer such as an ethylene-α olefin copolymer elastomer, or the like), or the like.

Further, the gasket 4 is disposed with a concave portion extending inward from a proximal end thereof. This recess portion has a female screw shape to be screwable with a male screw portion formed on an outer surface of a protruding portion 52 formed at a distal end of the plunger 5. As the recess portion and the male screw portion are screwed with each other, the plunger 5 is not removed from the gasket 4. The plunger 5 may be disposed in a detached state and attached at the time of use. In addition, the plunger 5 includes the protruding portion 52 protruding in a cylindrical shape forward from a disk portion at the distal end, and a male screw to be screwed with the recess portion of the gasket 4 is formed on the outer surface of the protruding portion. In addition, the plunger 5 includes a body portion 51 extending in an axial direction and having a cross-shaped cross-section and a disk portion 53 for pressing disposed at a proximal end.

As illustrated in FIGS. 4 to 6, the cap 3 includes the seal cap 7 and the cylindrical cover member 8.

The cap 3 is configured to be used in the state of being mounted to the barrel including: the barrel body portion 21; the nozzle portion 22 disposed at the distal end of the barrel body portion 21 and including the head portion 24 and the annular concave portion 25 formed at the proximal end of the head portion 24; and the puncture needle 6 including the puncture needle tip 61 at the distal end and the proximal end inserted into and fixed to the nozzle portion 22.

The seal cap 7 includes: the closed distal end portion 71a; the open proximal end portion 72; the hollow portion 70 including the puncture needle storage portion that stores the puncture needle 6; the insertion-allowing portion 73 into which the puncture needle tip 61 of the puncture needle 6 stored in the puncture needle storage portion is insertable; and the seal portion 75 in close contact with the nozzle portion to seal the puncture needle storage portion. The cylindrical cover member 8 is mounted on the outer side of the seal cap 7 and engages with the seal cap 7.

The seal cap 7 is made of the elastic material and is water vapor permeable for high-pressure steam sterilization.

As a material for forming the seal cap 7, at least the insertion-allowing portion 73 is made of an elastic material such that the puncture needle is insertable. As an elastic material that allows insertion of the puncture needle and includes water vapor permeable properties for high-pressure steam sterilization, a thermoplastic elastomer, a styrene elastomer such as an SBS elastomer and an SEBS elastomer, an olefin elastomer such as an ethylene-α-olefin copolymer elastomer, or the like is preferable. In addition, rubber such as butyl rubber, isoprene rubber, and silicone rubber may be used as the elastic material.

As illustrated in FIGS. 4, and 13 to 16, the seal cap 7 includes a cylindrical body portion 71 and a flange 77 extending from a proximal end of the cylindrical body portion 71 in the proximal end direction. The cylindrical body portion 71 has a form of an outer surface extending in a distal end direction by a predetermined length and having almost the same outer diameter or a diameter reduced slightly in the distal end direction. The flange 77 has an outer diameter larger than the outer diameter of the proximal end of the cylindrical body portion 71 and extends in the proximal end direction by a predetermined length with substantially the same outer diameter.

The cylindrical body portion 71 includes: the closed distal end portion 71a; the seal portion 75 that stores the distal end of the nozzle portion 22; the hollow portion 70 including the puncture needle storage portion that stores the puncture needle; the insertion-allowing portion 73 into which the puncture needle tip 61 of the puncture needle 6 stored in the hollow portion 70 is insertable; and a protruding portion 76 formed on an inner surface of the seal portion 75. Then, when the seal cap 7 is mounted to the nozzle portion 22 of the barrel 2, the puncture needle tip 61 is inserted into the insertion-allowing portion 73 of the seal cap 7 to be sealed, and further, the protruding portion 76 and the annular concave portion 25 of the nozzle portion 22 of the barrel 2 are engaged with each other so that the inner surface of the seal portion 75 and the outer surface of the nozzle portion 22 are brought into close contact with each other to form a sealed state of the puncture needle storage portion. The flange 77 includes an internal space to store a proximal-end-side portion of the nozzle portion 22, the proximal end 72, and a proximal end surface 72a.

Specifically, the seal cap 7 includes the protruding portion 76 disposed on the inner surface positioned on the distal end side of the open proximal end portion 72 by a predetermined length. The protruding portion 76 includes a vertex portion 76a protruding the most and an inclined portion (tapered portion) 76b extending in the distal end direction from the vertex portion 76a and having a protrusion height gradually decreasing in the distal end direction. In particular, in the present embodiment, the protruding portion 76 is an annular protruding portion, and the inclined portion 76b is a tapered portion in which an inner diameter of the seal portion 75 decreases in the distal end direction.

The inner diameter of the seal portion 75 at the vertex portion 76a is slightly smaller than the outer diameter of the distal end of the annular concave portion 25 of the nozzle portion 22 of the barrel 2. As a result, when the seal cap 7 is mounted to the nozzle portion 22 of the barrel 2, the protruding portion 76 and the annular concave portion 25 are engaged with each other. In addition, the distal-end-side inclined portion 76b extends to the distal end side of the annular concave portion 25 in a state in which the seal cap 7 is mounted to the nozzle portion 22 of the barrel 2. Further, the inner diameter of the seal portion 75 in the vicinity of at least the proximal end of the distal-end-side inclined portion 76b is slightly smaller than the outer diameter of the head portion 24 of the nozzle portion 22 of the barrel 2. As a result, when the seal cap 7 is mounted to the nozzle portion 22 of the barrel 2, the distal-end-side inclined portion 76b is pressed against the outer surface of the head portion 24 to be brought into close contact with the inclined portion 76b, and thus, undesirable removal of the seal cap 7 from the barrel 2 is further reduced.

In particular, in the present embodiment, the seal portion 75 of the seal cap 7 is in the state of being expanded outward by the nozzle portion 22 of the stored external cylinder 2 as illustrated in FIG. 4. Specifically, the seal portion 75 is in the state of being elastically deformed and slightly inflated. Thus, the seal portion 75 is formed such that a distance from the inner surface of the cylindrical cover member 8, which will be described later, is shorter than those of the other portions, thereby regulating inflow of an ozone gas under the atmospheric pressure at the time of manufacturing or the like.

In addition, the protruding portion 76 is formed to be annular along the inner surface of the seal portion 75 in the seal cap 7 of the present embodiment. Thus, the area of the distal-end-side inclined portion 76b, which is pressed against the outer surface of the head portion 24 be brought into close contact therewith increases as compared to the case where the protruding portions 76 are formed at intervals on the inner surface of the seal portion 75, and the undesirable removal of the seal cap 7 from the barrel 2 is further reduced. The protruding portions 76 may be formed at intervals on the inner surface of the seal portion 75.

In addition, in the seal cap 7 of the present embodiment, the protruding portion 76 further includes a proximal-end-side inclined portion 76c which extends in an open end (proximal end) direction from the vertex portion 76a and whose protruding height gradually decreases in the open end (proximal end) direction. As a result, when the seal cap 7 is mounted to the nozzle portion 22 of the barrel 2, the vertex portion 76a of the protruding portion 76 easily gets over the head portion 24 of the nozzle portion 22 from the distal end side.

In particular, in the present embodiment, the protruding portion 76 is the annular protruding portion, and the proximal-end-side inclined portion 76c is a proximal-end-side tapered portion in which the inner diameter of the seal portion 75 increases in the proximal end direction. In the seal cap 7 of the present embodiment, the proximal-end-side inclined portion (proximal-end-side tapered portion) 76c is shorter than the distal-end-side inclined portion (distal-end-side tapered portion) 76b and has a larger taper angle.

In addition, in the seal cap 7 of the present embodiment, the seal portion 75 includes a linear portion 76d extending in the distal end direction from the distal end of the distal-end-side inclined portion 76b of the protruding portion 76 by a predetermined length (specifically, to the proximal end of the hollow portion 70). The inner diameter of the seal portion 75 at the linear portion 76d is constant and slightly smaller than the outer diameter of the head portion 24 of the nozzle portion 22 of the barrel 2. Thus, when the seal cap 7 is mounted to the nozzle portion 22 of the barrel 2, the linear portion 76d is pressed against the outer surface of the head portion 24 to be brought into close contact therewith. The inner diameter of the seal portion 75 in the linear portion 76d may be larger than the outer diameter of the head portion 24 of the nozzle portion 22 of the barrel 2. In addition, the linear portion 76d may be omitted and the distal-end-side inclined portion 76b may be extended to the proximal end of the seal portion 75.

Further, a removal resistance of the seal cap 7 from the barrel 2 is preferably 1.5N to 20 N, and more preferably 5 to 8 N. As a result, it is possible to easily remove the seal cap 7 from the barrel 2 at the time of use of the prefilled syringe 1 while preventing the undesirable removal of the seal cap 7 from the barrel 2.

In addition, an inclination angle (taper angle) of the distal-end-side inclined portion 76b of the protruding portion 76 of the seal cap 7 is preferably 1 to 10 degrees, and more preferably 1 to 6 degrees. In addition, the protruding height of the vertex portion of the protruding portion 76 is preferably 0.1 to 0.5 mm, and more preferably 0.05 to 0.25 mm.

In addition, in the present embodiment, the proximal end of the distal-end-side inclined portion 76b of the protruding portion 76 of the seal cap 7 is positioned around the annular concave portion 25 of the nozzle portion 22 of the barrel 2, and the inner diameter of the seal portion 75 at least in the vicinity of the proximal end of the distal-end-side inclined portion 76b is slightly smaller than the outer diameter of the annular concave portion 25. In addition, the annular concave portion 25 of the barrel 2 is formed of the tapered reduced diameter portion disposed at the proximal end of the head portion 24 and whose diameter decreases in the proximal end direction in the present embodiment. As a result, when the seal cap 7 is removed from the barrel 2, the protruding portion 76 of the seal cap 7 is expanded outward along the annular concave portion 25 to easily get over the head portion 24.

Further, a length, in the axial direction of the head portion 24, of a portion of the distal-end-side inclined portion 76b of the protruding portion 76 of the seal cap 7, the portion pressed against the outer surface of the head portion 24 to be brought into close contact therewith is preferably 0.1 to 2.0 mm, and more preferably 0.3 to 1.5 mm. As a result, it is possible to reduce the undesirable removal of the seal cap 7 from the barrel 2, and to suppress the removal resistance of the seal cap 7 from the barrel 2 from being unnecessarily large.

Further, the seal cap 7 of the present embodiment includes a nozzle portion introduction portion 78 which is formed from the open proximal end portion 72 of the seal cap 7 to the proximal end of the seal portion 75 (the protruding portion 76) and extends with substantially the same inner diameter. The nozzle portion introduction portion 78 has the inner diameter slightly larger than the maximum inner diameter of the seal portion 75 and is slightly larger than the outer diameter of the head portion 24 of the nozzle portion 22 of the barrel 2. Thus, the seal cap 7 functions as an introduction portion of the nozzle portion 22 when the seal cap 7 is mounted to the nozzle portion 22 of the barrel 2. In addition, the nozzle portion introduction portion 78 includes an annular rising surface 79 erected toward the open proximal end portion 72 at a boundary with the proximal end of the seal portion 75 (the protruding portion 76).

Accordingly, when the distal end of the barrel 2 is inserted into the nozzle portion introduction portion 78 of the seal cap 7, the nozzle portion 22 of the barrel 2 has entered the inside of the nozzle portion introduction portion 78, and then, the annular distal end surface of the head portion 24 of the nozzle portion 22 abuts against the annular rising surface 79. In this state, the puncture needle 6 becomes substantially parallel to a central axis of the seal cap 7 and is in the state of entering the inside of the hollow portion 70.

In addition, the seal cap 7 of the present embodiment is configured such that the puncture needle tip 61 of the puncture needle 6 enters the inside of the hollow portion 70 not to reach the insertion-allowing portion 73 in the state in which the nozzle portion 22 of the barrel 2 is inserted into the seal cap and the annular distal end surface of the head portion 24 of the nozzle portion 22 abuts against the annular rising surface 79 of the nozzle portion introduction portion 78 of the seal cap 7.

In addition, the flange 77 protruding annularly outward is formed at the proximal end of the seal cap 7. A distal-end-side position of the flange 77 is positioned on the distal end side of the annular rising surface 79 of the hollow portion 70 and positioned in the vicinity of the vertex portion 76a of the protruding portion 76 (slightly closer to the proximal end opening portion 72 side than the vertex portion 76a in the case illustrated in FIGS. 4 and 14).

Further, the open proximal end portion 72 of the seal cap 7 includes the annular proximal end surface 72a, and a plurality of protrusions 74 is disposed on the proximal end surface 72a. Further, the proximal end surface 72a forms an engagement portion on the seal cap side at the time of engagement with the cylindrical cover member 8. The proximal end surface 72a also serves as a proximal end surface of the flange 77. Accordingly, it can be also said that the cap-side engagement portion is formed by the proximal end surface 72a and the flange 77. The proximal end surface of the flange 77 may be disposed on the distal end side of the open proximal end portion 72. In this case, the cap-side engagement portion is formed by not the proximal end surface 72a but the proximal end surface of the flange 77.

Next, the cylindrical cover member 8 will be described with reference to FIGS. 4 to 12.

The cylindrical cover member 8 is made of a thermoplastic plastic material harder than the seal cap 7, and is configured so that, by high-pressure steam sterilization of the syringe assembly 10, the force of engagement between the cylindrical cover member 8 and the seal cap 7 is increased compared to before sterilization.

As illustrated in FIGS. 4 to 12, the cylindrical cover member 8 is a cylindrical body including a seal cap storage portion 80 at the inside thereof. The cylindrical cover member 8 of the present embodiment includes a side surface covering portion 81 that covers a side surface of the seal cap 7 and a distal end covering portion 82 that covers the closed distal end portion of the seal cap 7.

Further, the cylindrical cover member 8 includes an engagement mechanism for regulation of removal which is configured in cooperation with the seal cap 7. In the present embodiment, the cylindrical cover member 8 includes an internal protruding portion disposed on the inner surface of the cylindrical cover member 8 to be engaged with the seal cap 7. Further, this internal protruding portion is displaced inward by heat of high-pressure steam sterilization, whereby the force of engagement between the cylindrical cover member 8 and the seal cap 7 is increased compared to before sterilization. In addition, the internal protruding portion extends obliquely from the inner surface of the cylindrical cover member 8 in a center axis direction of the cylindrical cover member 8 and in the distal end direction, and is elastically deformable. When the cylindrical cover member 8 is mounted to the seal cap 7, the internal protruding portion is pushed by the outer surface of the seal cap and is elastically deformed in a direction away from the center axis of the cylindrical cover member 8, and thus, it is easy to mount the cylindrical cover member 8 to the seal cap 7. In addition, when the cap 3 is removed from the barrel 2, a distal end of the internal protruding portion is pushed by the seal cap 7 to be deformed in a direction approaching the central axis of the cylindrical cover member 8, and thus, it is difficult for the cylindrical cover member 8 to be removed from the seal cap 7. In addition, a plurality of the internal protruding portions is disposed at intervals about the central axis of the cylindrical cover member 8. Specifically, the cylindrical cover member 8 of the present embodiment includes a plurality of internal protruding portions 85 (85a and 85b) disposed on the inner surface of the proximal end of the side surface covering portion 81. In particular, in the illustrated embodiment, the plurality of elastically-deformable internal protruding portions 85a and 85b extend obliquely from the inner side of the proximal end (or substantially the proximal end) of the side surface covering portion 81 in the central axis direction and in the distal end direction of the cylindrical cover member 8, and include free ends at positions that do not reach the center of the cylindrical cover member 8. Further, the free ends of the respective internal protruding portions 85 (85a and 85b) are capable of abutting against the proximal end surface 72a or the protrusion 74 of the seal cap 7.

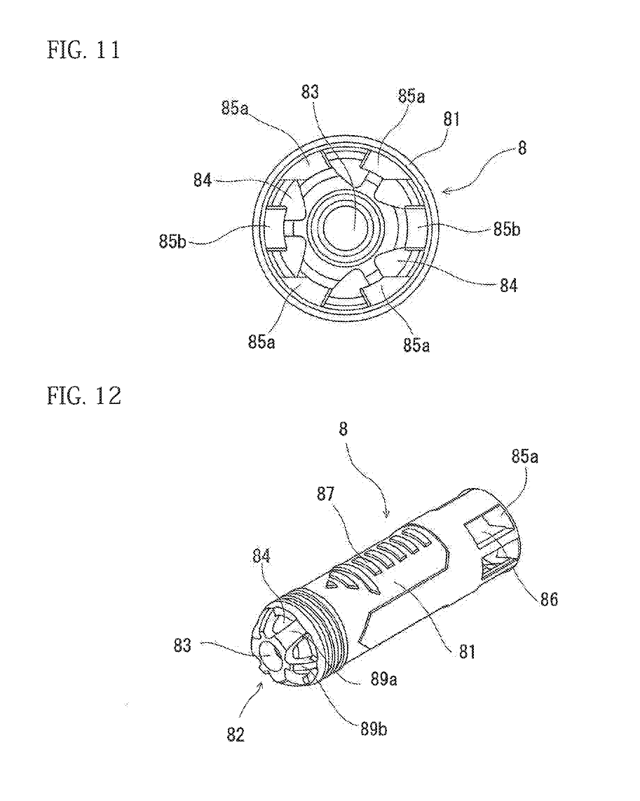

Further, a width of each of the four internal protruding portions 85a is narrowed toward the free end as illustrated in FIG. 11. In particular, the internal protruding portion 85a has one side surface being inclined so that the width is narrowed toward the free end. In addition, the two internal protruding portions 85a and 85b disposed so as to face each other extend to the free ends with the same width. In addition, an inner surface of the free end of each of the internal protruding portions 85a and 85b of the cylindrical cover member 8 is preferably an arcuate surface having the central axis of the cylindrical cover member 8 substantially as the center. Further, in the present embodiment, the free ends of the internal protruding portions 85a and 85b have circular arc portion and can reliably abut against the proximal end surface 72a or the protrusion 74 of the seal cap 7.

In addition, when the cylindrical cover member 8 is mounted on the seal cap 7, the free ends of the internal protruding portions 85a and 85b are deformed in the direction away from the center axis of the cylindrical cover member. In addition, when the cylindrical cover member 8 moves in the direction to be removed from the seal cap 7, the free ends of the internal protruding portions 85a and 85b are deformed in the direction approaching the central axis of the cylindrical cover member. Further, in the cylindrical cover member 8 of the present embodiment, rear-end-side surfaces of the plurality of internal protruding portions 85a and 85b of the cylindrical cover member 8 are inclined surfaces that are smooth and face toward the center of the cylindrical cover member, and form a guide portion configured to guide the seal cap 7. In addition, in the cylindrical cover member 8 of the present embodiment, the distal-end-side surfaces of the plurality of internal protruding portions 85a and 85b of the cylindrical cover member 8 are also inclined surfaces that are smooth and face toward the center of the cylindrical cover member. Further, each of the internal protruding portions 85a and 85b becomes thinner toward the free end, and has elastic deformability that is increased toward the free end.

Further, an outer diameter of the maximum outer diameter portion (flange) of the seal cap 7 is larger than a diameter of a circle formed by the inner surface of the free end of each of the internal protruding portions 85a and 85b of the cylindrical cover member 8 preferably by 0.5 to 5 mm, and more preferably by 1.5 to 3 mm. In addition, it is preferable that the inner diameter of the proximal end of the side surface covering portion 81, in other words, the inner diameter of the portion storing the flange 77 of the seal cap 7, be substantially equal to the outer diameter of the flange 77 of the seal cap 7. In particular, the proximal end of the side surface covering portion 81 is preferably in close contact with the flange 77 of the seal cap 7 after the high-pressure steam sterilization. In particular, the proximal end of the side surface covering portion 81 is preferably in close contact with and pressed against the flange 77 of the seal cap 7 after the high-pressure steam sterilization.

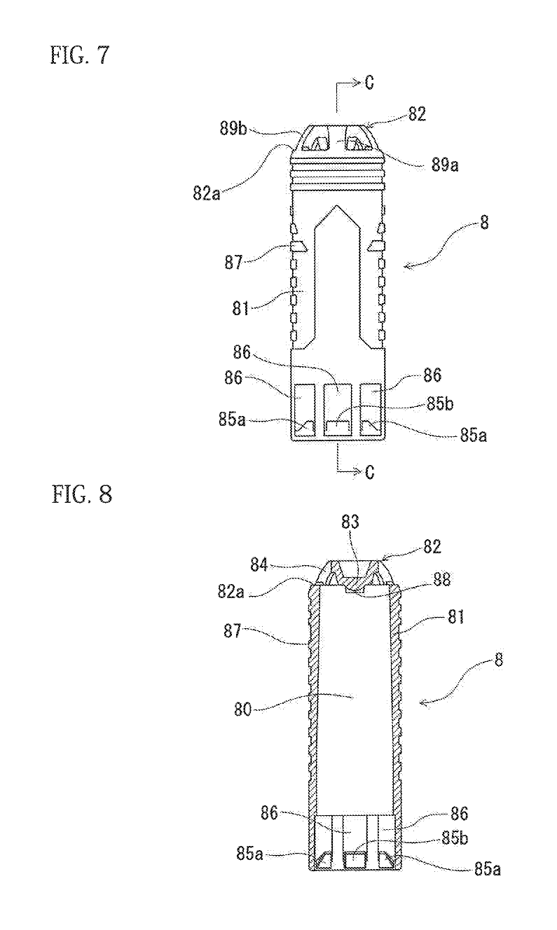

Further, in the cylindrical cover member 8 of the present embodiment, an opening 86 extending in the distal end direction from the proximal end of each of the internal protruding portions 85a and 85b is provided as illustrated in FIGS. 7 to 9. As a result, the internal protruding portions 85a and 85b are more easily deformed in the direction away from the center axis of the cylindrical cover member 8, and it is easy to mount the cylindrical cover member 8 to the seal cap 7. Further, the six internal protruding portions (specifically, the four internal protruding portions 85a and the two internal protruding portions 85b) are arranged at substantially equal angles with respect to the central axis of the cylindrical cover member 8 in the present embodiment.

Further, the cylindrical cover member 8 of the present embodiment includes a through-hole 84 disposed at the distal end, specifically, in the distal end covering portion 82 as illustrated in FIGS. 4 to 12. Further, the cap 3 includes a steam guide gap 32 that is formed by the inner surface of the cylindrical cover member 8 and the outer surface of the seal cap 7, communicates with the through-hole 84, and extends to the proximal end of the puncture needle storage portion of the seal cap 7 (in other words, the distal end of the flange). In the present embodiment, the steam guide gap 32 extends from the through-hole 84 to the distal end of the flange 77 of the seal cap 7. In addition, the steam guide gap 32 is an annular space so as to surround the outer surface of the seal cap 7.

In addition, the steam guide gap 32 communicates with the opening 86 at a proximal end 32a thereof. Specifically, a part of the proximal end (in other words, a portion closer to the distal end side than the flange 77) of the cylindrical body portion 71 of the seal cap 7 is positioned at the distal end of the opening 86, and in this portion, a gap 32a is formed between the outer surface of the seal cap 7 (specifically, the outer surface of the proximal end of the cylindrical body portion 71 of the seal cap 7) and the inner surface of the proximal end of the cylindrical cover member 8. As a result, the steam guide gap 32 has the distal end communicating with the through-hole 84, and the proximal end communicating with the opening 86. Thus, the flow of steam for sterilization becomes favorable. The proximal end 32a of the steam guide gap 32 may not necessarily communicate with the opening 86. In this case, the flow of the steam for sterilization is secured due to a pressure fluctuation at the time of high-pressure steam sterilization.

Further, in the present embodiment, the seal portion 75 of the seal cap 7 is in the state of being expanded outward by the nozzle portion 22 of the stored external cylinder 2 as illustrated in FIG. 4. Specifically, the seal portion 75 is in the state of being elastically deformed and slightly inflated. Thus, the steam guide gap in this portion is a narrow portion 32b having a narrower width than the other portion of the steam guide gap 32, and regulates inflow of an ozone gas under atmospheric pressure at the time of manufacturing or the like.

The width of the steam guide gap 32 (a distance between the inner surface of the cylindrical cover member 8 and the outer surface of the seal cap 7) is preferably 0.1 to 2 mm. The flowability of the steam for sterilization deteriorates if the width of the steam guide gap 32 is smaller than 0.1 mm, and wobbling of the cylindrical cover member 8 with respect to the seal cap 7 is large so that the operability at the time of detaching the cap 3 from the barrel 2 deteriorates if the width is larger than 2 mm. In addition, the narrow portion 32b is narrower than the width of the other portion of the steam guide gap 32 (the distance between the inner surface of the cylindrical cover member 8 and the outer surface of the seal cap 7) preferably by 0.1 to 2 mm.

Further, in the cylindrical cover member 8 of the present embodiment, the distal end covering portion 82 includes a plurality of frames connected to the distal end of the side surface covering portion 81, and the through-hole 84 is formed between adjacent frames. More specifically, the distal end covering portion 82 includes: a distal end central portion 83 including a concave portion at the center; the plurality of frames connecting the distal end central portion 83 and the distal end of the side surface covering portion 81; and the plurality of through-holes 84 each of which is formed between the frames. In particular, the distal end covering portion 82 includes six frames 89a and 89b in the present embodiment. Further, the frame 89a is thick, and the frame 89b is thin. In addition, the frames 89a and 89b are alternately arranged three by three. In addition, all the frames 89a and 89b are disposed so as to be substantially equiangular with respect to the center axis of the cylindrical cover member 8. In addition, the number of the through-holes is six in the present embodiment, but is preferably about 2 to 10 and more preferably 3 to 8. In addition, the total area of the through-holes in a plan view of the distal end side of the cylindrical cover member 8 is preferably 3 to 25 mm², and more preferably 5 to 20 mm². With such a configuration, the distal end maintains sufficient strength and allows favorable passage of the steam for sterilization therethrough.

Further, in the cylindrical cover member 8, a corner portion 82a formed at a boundary portion between the proximal ends of the frames 89a and 89b and the distal end of the side surface covering portion 81 and faces the distal end direction in a side view illustrated in FIG. 7. A width of the corner portion 82a is preferably 0.2 to 1 mm. As such a corner portion 82a is disposed, the syringe assembly 10 is easily inserted into a cylindrical portion 142 of the barrel holding member 104 to be described later or a hole of a centering plate to be used at the time of filling a drug solution while securing a size of the through-hole. In addition, the cylindrical cover member 8 includes multiple anti-slip ribs 87 formed on an outer surface as illustrated in the drawings.

Further, the cylindrical cover member 8 includes an opposing inner surface 88 that opposes the distal end surface of the closed distal end portion 71a of the seal cap 7 and a gap 31 is formed between the opposing inner surface 88 and the closed distal end portion 71a in the cap 3 of the present embodiment as illustrated in FIGS. 4 and 6. Thus, the flow of water vapor inside the cap becomes favorable. In addition, condensed water at the time of sterilization is hardly trapped so that a drying property becomes favorable. In particular, at the time of performing sterilization with the distal end of the cap 3 facing downward, condensed water tends to collect at the tip portion, which is effective. A length of the gap 31, in other words, a distance between the opposing inner surface 88 and the closed distal end portion 71a is preferably 0.1 to 5 mm, and more preferably 0.3 to 2 mm.

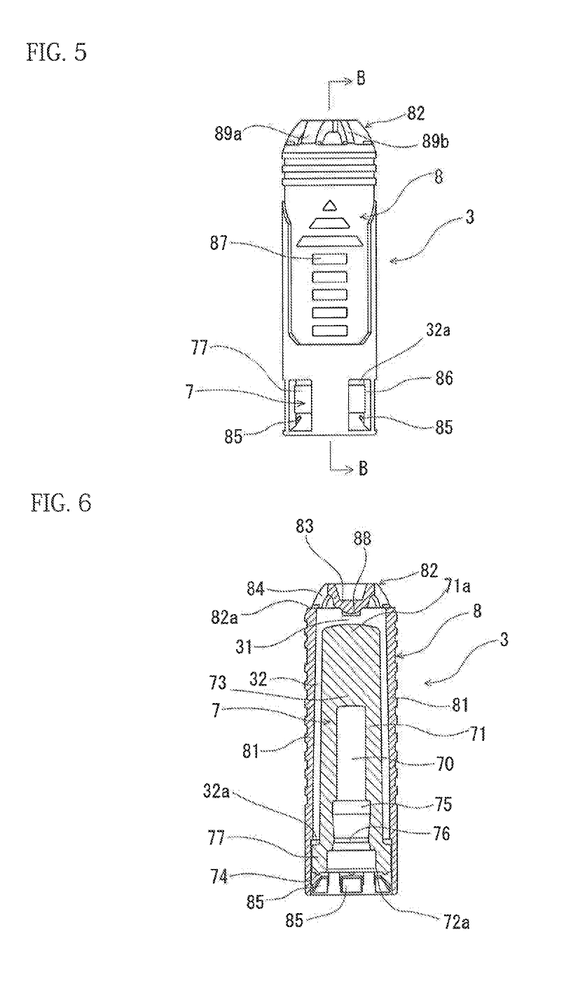

In the above-described structural characteristics of the cylindrical cover member 8, the syringe assembly (in other words, the barrel with a puncture needle, to which the cap is mounted) 10 has an excellent effect regardless of whether the force of engagement between the cylindrical cover member 8 and the seal cap 7 after the high-pressure steam sterilization is increased compared to before sterilization or not. That is, for example, in the cap 3 illustrated in FIGS. 5 and 6, the cylindrical cover member 8 is engaged with the proximal end (flange 77) of the seal cap 7 at the proximal end, and thus, has a sufficient force of engagement even before sterilization. Further, the cap 3 includes the gap 32 formed between the cylindrical cover member 8 and the seal cap 7 and the through-hole 84 communicating with the gap 32. Thus, in the case of EOG (ethylene oxide gas) sterilization, because an ethylene oxide gas flows from the through-hole 84 to the gap 32, the gas flowability is favorable. In addition, the width of the corner portion formed at the distal end of the cylindrical cover member is 0.2 to 1 mm as described above, and thus, the syringe assembly can be easily inserted into the cylindrical portion of the cylindrical cover member or the hole of the centering plate to be used at the time of filling the drug solution while securing the size of the through-hole 84.

In addition, in the case where the cap is of the above-described type, it is preferable that the cylindrical cover member 8 and the seal cap 7 have a sufficient force of engagement before sterilization. In addition, in this case, a sterilization method other than the high-pressure steam sterilization, such as EOG sterilization or radiation sterilization may be used as the sterilization method. The seal cap 7 may be one that is not permeable to water vapor (such as steam) for high-pressure steam sterilization. In addition, in the case of using the EOG sterilization, the seal cap 7 is permeable to ethylene oxide gas.

As a material for forming the cylindrical cover member 8, a material harder than the material for forming the seal cap 7 is used. Examples of the material may include various resins such as polyesters such as polypropylene, polyethylene, polystyrene, polyamide, polycarbonate, polyvinyl chloride, poly (4-methylpentene-1), an acrylic resin, an acrylonitrile-butadiene-styrene copolymer, polyester such as polyethylene terephthalate, and cyclic polyolefins.

Further, the cylindrical cover member 8 preferably has heat shrinkability depending on heat during the high-pressure steam sterilization. The cap 3 thermally shrinks due to the heat during high-pressure steam sterilization. As a result, the force of engagement between the cylindrical cover member 8 and the seal cap 7 is increased more than before sterilization. As long as the form of the cylindrical cover member 8 at the time of molding includes the gap 32 between the cylindrical cover member 8 and the outer surface of a portion of the seal cap 7 excluding the flange 77 as illustrated in FIGS. 4 and 6, the gap 32 which communicates with the through-hole 84 and through which water vapor during the high-pressure steam sterilization can pass is maintained between the inner surface of the cylindrical cover member 8 and the outer surface of the seal cap 7 as illustrated in FIG. 4.

Further, the cylindrical cover member 8 is preferably mounted to the seal cap 7 after the seal cap 7 is mounted to the barrel 2. When only the seal cap is mounted at an early stage in this manner, it can be confirmed whether the puncture needle is inserted straight into the seal cap. The seal cap bends if the puncture needle is inserted obliquely, and thus, the state of the puncture needle can be easily confirmed. However, the cap does not bend in the cap to which the cylindrical cover member has been mounted even when the needle is bent and inserted so that it is difficult to confirm the state of the puncture needle. Further, the syringe assembly 10 is preferably subjected to the high-pressure steam sterilization.

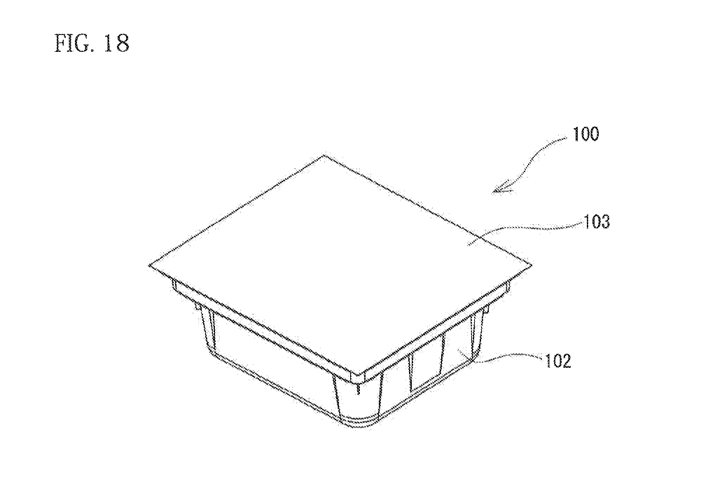

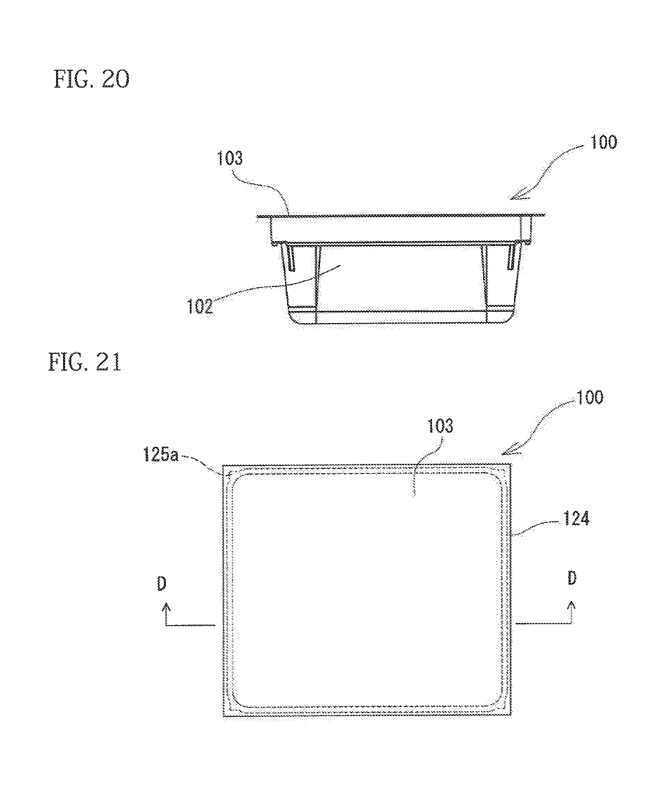

Next, an embodiment of a packaging storing a plurality of the syringe assemblies will be described with reference to FIGS. 18 to 22.

A sterilizable or sterilized prefilled syringe assembly packaging 100 storing the plurality of syringe assemblies of the present invention includes: a container 102 having an open upper surface and having shape retainability; an external cylinder holding member 104 capable of holding the plurality of syringe assemblies 10 stored in the container 102; the plurality of syringe assemblies 10 held by the barrel holding member 104; and a sheet-shaped lid member 103 which hermetically seals the open upper surface of the container 102 and is peelable.

The prefilled syringe assembly packaging 100 of the present invention is a sterilizable or sterilized prefilled syringe assembly packaging. High-pressure steam sterilization is used as a sterilization method.

As illustrated in FIGS. 18, 19, and 22, the prefilled syringe assembly packaging 100 of the present embodiment includes: the container 102; the barrel holding member 104 capable of holding the plurality of syringe assemblies 10; the plurality of syringe assemblies 10 held by the barrel holding member 104; and the sheet-shaped lid member 103 which hermetically seals the open upper surface of the container 102 and is peelable. Further, the packaging 100 includes a ventilation portion disposed on the container 102 or the sheet-shaped lid member 103 and including bacterial impermeable properties and sterilization gas flowability.

As illustrated in FIGS. 18 to 22, the container 102 is of a tray-shaped container having a predetermined depth and a certain degree of strength and shape retainability, and includes: a body portion 121, an external cylinder holding member holding portion 126 formed at an upper portion of the body portion 121 and configured to hold a peripheral edge of the barrel holding member 104 holding the plurality of syringe assemblies 10, and an annular flange 124 disposed on the open upper surface.

Further, an annular heat-sealing convex portion 125 for fixing with the sheet-shaped lid member 103 is disposed on an upper surface of the annular flange 124. Further, the barrel holding member holding portion 126 is formed at a position on a bottom surface side by a predetermined length from the flange 124. In the container 102 of this embodiment, the barrel holding member holding portion 126 is an annular step portion such that the peripheral edge of the barrel holding member 104 holding the plurality of syringe assemblies 10 can be placed.

The container 102 preferably has a certain degree of shape retainability and rigidity. In addition, it is desirable to use a thermoplastic material having heat resistance (120° C. or higher) in order to cope with the high-pressure steam sterilization. As Examples of a material having a certain degree of shape retainability, a certain degree of rigidity, heat resistance, and thermoplasticity include polyolefin such as polypropylene and polyethylene, a vinyl chloride resin, a polystyrene/polypropylene resin, polyethylene/ionomer (for example, ethylene-based, styrene-based, fluorine-based)/polyethylene, a polyester resin (for example, polyethylene terephthalate, polybutylene terephthalate, amorphous polyethylene terephthalate), and PP/EVOH/PP (laminate). In this case, a thickness of the container 102 is preferably about 0.05 to 4.00 mm, and particularly, more preferably 1.00 to 2.00 mm.

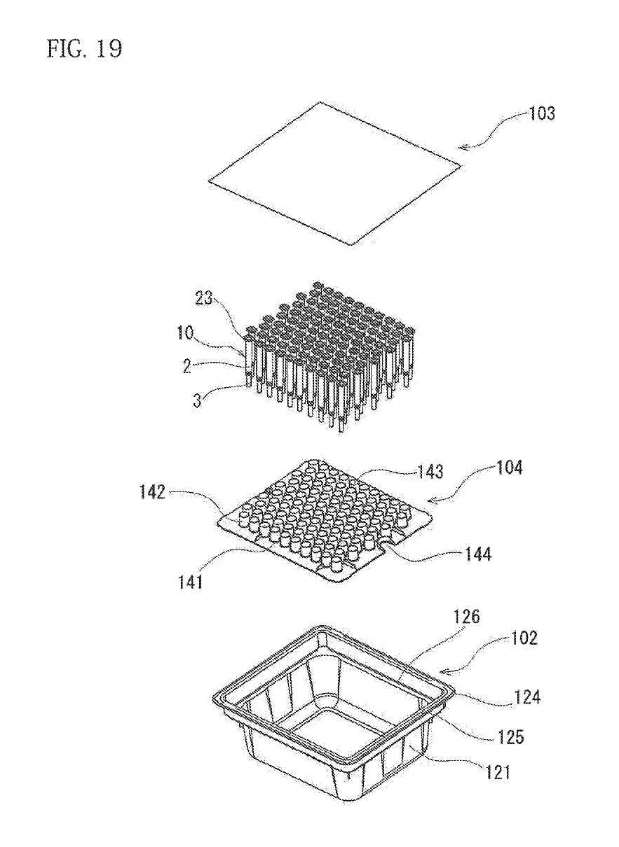

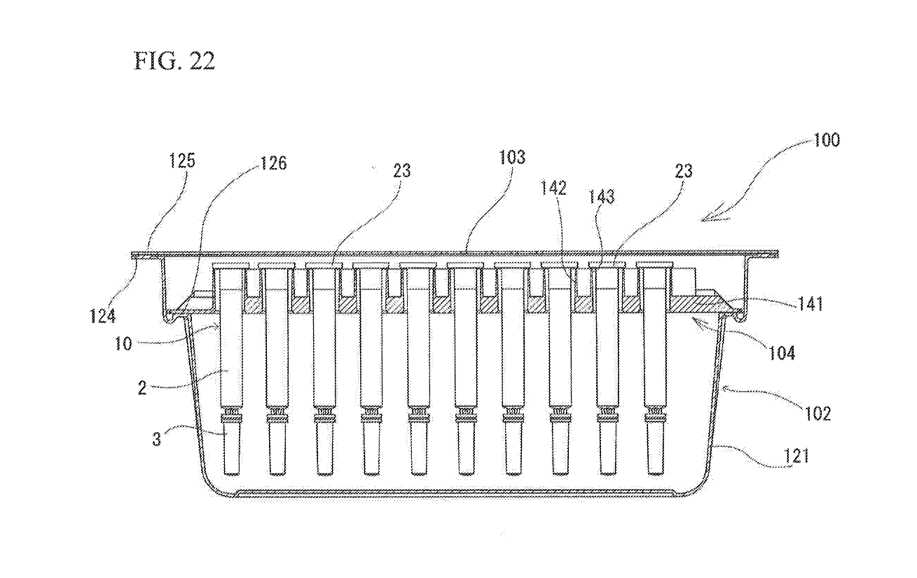

As illustrated in FIGS. 19 and 22, the barrel holding member 104 capable of holding the plurality of syringe assemblies 10 includes a substrate portion 141 and a plurality of cylindrical portions 142 protruding upward from the substrate portion 141. Further, an external cylinder holding opening 143 is formed in the cylindrical portion 142 and a cutout portion 144 for grip is formed on a side portion of the substrate portion 141. An inner diameter of the cylindrical portion 142 and the barrel holding opening 143 is larger than an outer diameter of the largest diameter portion of the syringe assembly 10 to be held, and is set to prevent passage of the flange portion 23 of the syringe assembly 10 to be held.

Thus, the syringe assembly 10 penetrates through the cylindrical portion 142 and the flange 23 of the syringe assembly 10 is in the state of being suspended by the barrel holding opening 143 as illustrated in FIG. 22. In addition, a lower end (the distal end of the seal cap 7) of the syringe assembly 10 held by the barrel holding member 104 is not in contact with a bottom surface of the container 102 as illustrated in FIG. 22. In other words, the bottom surface of the container 102 and the lower end of the syringe assembly 10 held by the barrel holding member 104 (the distal end of the seal cap 7) are separated from each other and do not inhibit the flow of water vapor. It is desirable that a material for forming the barrel holding member 104 also include heat resistance (120° C. or higher) in order to cope with the high-pressure steam sterilization.

As the sheet-shaped lid member 103, it is desirable to use a member to which microparticles such as bacteria and viruses are impermeable for high-pressure steam sterilization and water vapor is permeable. In addition, a member capable of heat-sealing of the container 102 is preferable. As the sheet-shaped lid member 103, for example, a synthetic resin nonwoven fabric, specifically, a nonwoven fabric made of a synthetic resin material such as polyolefin known as Tyvek®, a synthetic resin porous membrane, and the like can be suitably used.

Further, the sheet-shaped lid member 103 is heat-sealed against the heat-sealing convex portion 125 disposed on the annular flange 124 of the container 102 such that a peripheral edge thereof is peelable. An outer edge of the sheet-shaped lid member 103 is not heat-sealed against the annular flange 124 of the container 102 and is easily peeled off in the first embodiment. In addition, a protruding portion 125a disposed at a corner portion of the heat-sealing convex portion 125 functions as a peeling start portion. A thickness of the sheet-shaped lid member 103 is preferably about 0.05 to 1.00 mm, and more preferably about 0.10 to 0.50 mm.

The ventilation portion is disposed on the sheet-shaped lid member 103 in the above-described first embodiment, but the invention is not limited thereto, and the ventilation portion may be disposed on the container 102.

In one embodiment, a syringe assembly includes: an external cylinder that includes an external cylinder body portion, a nozzle portion disposed at a distal end of the barrel body portion, and a puncture needle including a puncture needle tip at a distal end of the puncture needle and having a proximal end fixedly inserted into the nozzle portion; and a cap mounted to the barrel and sealing the puncture needle tip. The cap includes: a seal cap that includes a closed distal end portion, an open proximal end portion, a hollow portion including a puncture needle storage portion storing the puncture needle, an insertion-allowing portion to which the puncture needle tip of the puncture needle stored in the puncture needle storage portion is insertable, and a seal portion in close contact with the nozzle portion to seal the puncture needle storage portion; and a cylindrical cover member mounted at an outer side of the seal cap to be engaged with the seal cap. The seal cap is made of an elastic material and includes water vapor permeable properties for high-pressure steam sterilization, and the cylindrical cover member is made of a thermoplastic plastic material harder than the seal cap, and a force of engagement between the cylindrical cover member and the seal cap is increased compared to before sterilization by autoclave sterilization of the syringe assembly.

In this syringe assembly, the force of engagement between the cylindrical cover member and the seal cap is increased compared to before sterilization in the case of selecting the sterilization accompanied by heating such as the high-pressure steam sterilization. Thus, when the cap is removed at the time of use, only the cylindrical cover member is removed from the barrel, and it is possible to reliably remove the entire cap without the sea cap remaining on the barrel.

In one aspect, the force of engagement is increased compared to before the sterilization due to deformation of the cylindrical cover member caused by heat during the autoclave sterilization.

In one aspect, the cylindrical cover member includes an internal protruding portion disposed on an inner surface of the cylindrical cover member to be engaged with the seal cap, the internal protruding portion being displaced inward due to heat during the autoclave sterilization such that the force of engagement is increased compared to before the sterilization.

In one aspect, the internal protruding portion extends obliquely in a center axis direction and a distal end direction of the cylindrical cover member and is elastically deformable.

In one aspect, the cylindrical cover member includes a plurality of internal protruding portions disposed on an inner surface of the cylindrical cover member to be engaged with the seal cap and disposed at intervals about a central axis of the cylindrical cover member, and the cylindrical cover member includes openings extending in a distal end direction from a proximal end of each of the plurality of internal protruding portions.

In one aspect, the cylindrical cover member includes: a side surface covering portion that covers a side surface of the seal cap; and a distal end covering portion that covers the closed distal end portion of the seal cap, in which the distal end covering portion includes an opposing inner surface that opposes a distal end surface of the closed distal end portion of the seal cap, and in which a gap is formed between the opposing inner surface and the closed distal end portion.

In one aspect, the distal end covering portion of the cylindrical cover member includes a plurality of frames connected to a distal end of the side surface covering portion, and a through-hole is formed between the frames adjacent to each other.

In one aspect, in a side view, the cylindrical cover member includes a corner portion that is formed at a boundary between a proximal end of the frame and a distal end of the side surface covering portion and that faces the distal end direction.

In one aspect, the cylindrical cover member includes a through-hole disposed at a distal end, and the cap includes a steam guide gap that is formed by an inner surface of the cylindrical cover member and an outer surface of the seal cap, communicates with the through-hole, and extends to a proximal end of the puncture needle storage portion of the seal cap.

In one aspect, the seal portion of the seal cap stores the nozzle portion and is in a state of being expanded outward by a distal end of the stored nozzle portion, and the steam guide gap of the seal portion has a narrower gap distance than other portions.

In one aspect, the cylindrical cover member has been attached to the seal cap after the seal cap has been attached to the barrel.

In one aspect, the syringe assembly has been subjected to high-pressure steam sterilization.

In another embodiment, a prefilled syringe including: the syringe assembly described above; a gasket stored in the barrel and slidable in the barrel in a liquid-tight manner; and a medical solution filled in a space formed by the barrel and the gasket.

In another embodiment, a syringe assembly packaging storing a plurality of the syringe assemblies described above includes: a container having an open upper surface and having shape retainability; an external cylinder holding member capable of holding the plurality of syringe assemblies; the plurality of syringe assemblies held by the barrel holding member; a sheet-shaped lid member that hermetically seals the open upper surface of the container and is peelable; and a ventilation portion that is disposed on the container or the lid member and includes bacterial impermeable properties and sterilization gas flowability, and subjected to high-pressure steam sterilization.

What is claimed is:

1. A syringe assembly comprising:
a barrel comprising:
a barrel body portion,
a nozzle portion disposed at a distal end of the barrel body portion, and
a puncture needle including a puncture needle tip at a distal end of the puncture needle and having a proximal end fixed in the nozzle portion; and
a cap mounted to the barrel and sealing the puncture needle tip, the cap comprising:
a seal cap that includes a closed distal end portion, an open proximal end portion, a hollow portion including a puncture needle storage portion storing the puncture needle, an insertion-allowing portion into which the puncture needle tip of the puncture needle stored in the puncture needle storage portion is insertable, and a seal portion in contact with the nozzle portion to seal the puncture needle storage portion, and
a cylindrical cover member mounted at an outer side of the seal cap;
wherein the seal cap is made of an elastic material and is permeable to water vapor permeable for high-pressure steam sterilization, and the cylindrical cover member is made of a thermoplastic material harder than the seal cap; and
wherein the seal cap and cylindrical cover member are configured such that a force of engagement between the cylindrical cover member and the seal cap is increased by autoclave sterilization of the syringe assembly.

2. The syringe assembly according to claim 1, wherein the seal cap and cylindrical cover member are configured such that the force of engagement between the cylindrical cover member and the seal cap is increased by heat during the autoclave sterilization.

3. The syringe assembly according to claim 1, wherein the cylindrical cover member comprises an internal protruding portion disposed at an inner surface of the cylindrical cover member to be engaged with the seal cap, the internal protruding portion being configured to be displaced inward due to heat during the autoclave sterilization to increase the force of engagement between the cylindrical cover member and the seal cap.

4. The syringe assembly according to claim 3, wherein the internal protruding portion extends obliquely in a center axis direction and a distal end direction of the cylindrical cover member and is elastically deformable.

5. The syringe assembly according to claim 1, wherein:
the cylindrical cover member comprises a plurality of internal protruding portions disposed at an inner surface of the cylindrical cover member, the internal protruding portions being configured to be engaged with the seal cap and being disposed at intervals about a central axis of the cylindrical cover member; and
the cylindrical cover member comprises openings extending in a distal end direction from a proximal end of each of the plurality of internal protruding portions.

6. The syringe assembly according to claim 1, wherein:
the cylindrical cover member comprises:
a side surface covering portion that covers a side surface of the seal cap, and
a distal end covering portion that covers the closed distal end portion of the seal cap;
the distal end covering portion includes an inner surface that opposes a distal end surface of the closed distal end portion of the seal cap; and
a gap is formed between the inner surface of the distal end covering portion and the closed distal end portion of the seal cap.

7. The syringe assembly according to claim 6, wherein:
the distal end covering portion of the cylindrical cover member comprises a plurality of frames connected to a distal end of the side surface covering portion; and
a through-hole is formed between adjacent ones of the frames.

8. The syringe assembly according to claim 7, wherein, in a side view, the cylindrical cover member comprises a corner portion that is formed at a boundary between a proximal end of the frame and a distal end of the side surface covering portion and that faces a distal end direction.

9. The syringe assembly according to claim 1, wherein:
the cylindrical cover member includes a through-hole disposed at a distal end of the cylindrical cover member; and
the cap comprises a steam guide gap that is formed by an inner surface of the cylindrical cover member and an outer surface of the seal cap, the steam guide gap communicating with the through-hole and extending to a proximal end of the puncture needle storage portion of the seal cap.

10. The syringe assembly according to claim 9, wherein:
the seal portion of the seal cap stores the nozzle portion and is in a state of being expanded outward by a distal end of the stored nozzle portion; and
the steam guide gap has a narrower gap distance at the seal portion than at other portions of the cap.

11. The syringe assembly according to claim 1, wherein the cylindrical cover member has been attached to the seal cap after the seal cap has been attached to the barrel.

12. The syringe assembly according to claim 1, wherein the syringe assembly has been subjected to high-pressure steam sterilization.

13. A prefilled syringe comprising:
the syringe assembly according to claim 1;
a gasket stored in the barrel and slidable in the barrel in a liquid-tight manner; and
a medical solution filled in a space formed by the barrel and the gasket.

14. A syringe assembly packaging storing a plurality of the syringe assemblies according to claim 1, the packaging comprising:
a container having an open upper surface and having shape retainability;
a barrel holding member holding a plurality of syringe assemblies according to claim 1;
a sheet-shaped lid member that hermetically seals the open upper surface of the container and is peelable; and
a ventilation portion that is disposed on the container or the lid member and is bacterial impermeable and configured to allow sterilization gas to flow therethrough;
wherein the packaging has been subjected to high-pressure steam sterilization.
